(19) United States
(12) Patent Application Publication
(10) Pub. No.: US 2025/0090431 A1
(43) Pub. Date: Mar. 20, 2025

(54) SILICONE FREE COSMETIC COMPOSITION

(71) Applicant: SHISEIDO COMPANY, LTD., Tokyo (JP)

(72) Inventors: Binhua YANG, East Windsor, NJ (US); Nancy HANNA, East Windsor, NJ (US)

(73) Assignee: SHISEIDO COMPANY, LTD., Tokyo (JP)

(21) Appl. No.: 18/368,437

(22) Filed: Sep. 14, 2023

(51) Int. Cl. A61K 8/06; A61K 8/31; A61K 8/34; A61K 8/55; A61K 8/81; A61K 8/92; A61Q 5/00; A61Q 19/00

(52) U.S. Cl. CPC A61K 8/062; A61K 8/31; A61K 8/345; A61K 8/553; A61K 8/8152; A61K 8/8182; A61K 8/922; A61Q 5/00; A61Q 19/00; A61K 2800/34; A61K 2800/48; A61K 2800/594

(57) ABSTRACT

An oil-in-water emulsion cosmetic composition includes at least one superabsorbent polymer, at least one water soluble emollient, and at least one thickener.

### FIELD

The present disclosure relates in general to cosmetic compositions, such as oil-in-water type cosmetic compositions.

### SUMMARY

One embodiment is a cosmetic composition comprising: at least one superabsorbent polymer, at least one water soluble emollient, and at least one thickener, wherein the composition is an oil-in-water emulsion.

One embodiment is a cosmetic composition comprising: at least one superabsorbent polymer, at least one water soluble emollient, and at least two polymer thickeners.

### FIGURES

FIG. 1 presents results of texture mapping of formulations with different polymers.

FIG. 2 presents results of texture mapping of formulations with different hydrophilic emollients.

FIG. 3 provides flow profile comparison between the formulation according to one of the embodiments and commercially available formulations.

### DETAILED DESCRIPTION

Unless otherwise specified “a” or “an” means one or more.

As used herein, the term “about” placed before a specific numeric value may mean ±20% of the numeric value; ±18% of the numeric value, ±15% of the numeric value; ±12% of the numeric value; ±8% of the numeric value; ±5% of the numeric value; ±3% of the numeric value; ±2% of the numeric value; ±1% of the numeric value or ±0.5% of the numeric value. All the numeric values in this disclosure other than the numeric values in the working examples should be treated as preceded by the word “about.”

Unless otherwise specified, all content information for ingredients of compositions expressed as % refers to % by mass, relative to the total mass of the composition.

As used herein, the expression “oil-in-water emulsion” and related expressions refers to an oil-in-water type emulsified composition in which an oil phase is dispersed in an aqueous (water) phase.

In general, silicones can make cosmetics products feel more elegant and appealing to consumers. Silicones can impart a velvety texture when applying products on the skin. Silicones can also help with the slip and silky feel of a cosmetic product so that it spreads easily onto skin and allows foundation to blend smoothly as well. Silicone can also provide a smooth and flawless base for makeup, help makeup last longer, and help provide a blurred soft-focus effect.

Some consumers also have environmental concerns about a low biodegradability of silicone. Thus, eliminating silicone from cosmetic products may be desirable.

Furthermore, 1,4-dioxane and ethylene oxide impurities of polyethylene glycol (PEG) surfactants may present concerns for satisfying “clean beauty” requirements, such as safety, non-toxicity and transparency in labeling, for cosmetic products. Thus, eliminating PEG containing ingredients, such as PEG surfactants, from a cosmetic product may be desirable.

However, making a silicone-free cosmetic product that has the same texture and performance as silicone containing cosmetic products may be a challenging task. Such task may become even more challenging when a silicone-free cosmetic product is PEG-free, i.e. it does not contain PEG containing ingredients, such as PEG surfactants. Furthermore, such task may be more challenging when a silicone-free cosmetic product does not contain ethanol.

The present disclosure addresses such challenge(s).

The present disclosure provides a silicone-free cosmetic composition, which may have a silicone-like texture and performance, i.e., texture and performance, which are similar to a silicone containing cosmetic composition, without containing silicone. For example, the silicone-free cosmetic composition may have sensory properties, such as silkiness, softness, weightlessness and/or cushiony feel, similar to the ones of a cosmetic composition containing silicone. Also, the texture of the silicone-free cosmetic composition may be similar to the one of a silicone containing cosmetic composition.

In one embodiment, a silicone-free cosmetic composition may include at least one superabsorbent polymer, at least one water soluble emollient, and at least one thickener, while being an oil-in-water emulsion.

In another embodiment, a silicone-free cosmetic composition may include at least one superabsorbent polymer, at least one water soluble emollient, and at least two polymer thickeners.

In some embodiments, the silicone-free cosmetic composition may be free of any polyethylene glycol containing ingredients, such as PEG-containing surfactants.

In some embodiments, the silicone-free cosmetic composition does not contain an unsubstituted mono-alcohol, such as methanol, ethanol, propanol or isopropanol.

In some embodiments, the cosmetic composition may have a viscosity at 10 rpm (rotations per minute) from 15000 cPs to 40000 cPs or 20000 cPs to 35000 cPs. In some embodiments, the cosmetic composition may have a viscosity at 10 rpm from 30000 cPs to 40000 cPs or from 31000 cPs to 39000 cPs or from 32000 cPs to 38000 cPs or from 33000 cPs to 37000 cPs or from 34000 cPs to 36000 cPs.

The silicone-free cosmetic composition may be stable so that its viscosity does not vary by more than 20% or more than 18% or more than 16% or more than 14% or more than 12% or more than 10% when stored for weeks between storage temperatures from −5 C to 50 C.

In some embodiments, the cosmetic composition may have a pH from 5.0 to 7.0 or from 5.5 to 6.5 or from 5.2 to 6.2 or from 5.4 to 6.0 or from 5.6 to 6.0 or from 5.8 to 6.0.

The silicone-free cosmetic composition may be stable so that its pH does not vary by more than 12% or more than 10% or more than 8% when stored for 4 weeks between storage temperatures from −5 C to 50 C.

Applications of the composition are not limited to cosmetic applications, such as skin care products. For example, in some embodiments, applications of the cosmetic composition may include but are not limited to applications in a sun care product, in makeup and a color cosmetics product, such as foundation, powder, blush, eye shadow, etc.

In some embodiments, the cosmetic composition may be non-comedogenic.

In some embodiments, the cosmetic composition may feel lightweight when applied to a keratinous surface, such as a skin.

In some embodiments, the cosmetic composition may blend smoothly with a keratinous surface, such as a skin, after applying the composition to the keratinous surface.

The silicone-free cosmetic composition may have texture comparable to commercially available silicone containing cosmetic compositions.

In some embodiments, the cosmetic composition may be used alone. In other words, the composition may be applied alone, without another composition, to a keratinous surface or substrate, such as skin or hair, of a subject, such as a human. Yet, in some embodiments, the cosmetic composition may be used together with another composition, which may be, for example, a cosmetic or skincare composition, such as a makeup composition or another skin care product. For example, in certain embodiments, the cosmetic composition may be applied to the keratinous surface or substrate before or after another composition is applied.

Preferably, the cosmetic composition can be used as a primer, meaning that it may be deposited on the keratinous substrate as an initial product for delivering hydrating, plumping, blurring and/or long wear effect to the keratinous surface or substrate.

In some embodiments, the cosmetic composition may have one or multiple of unique sensory properties, such as those described above. In some embodiments, the cosmetic composition may provide a weightlessness, e.g., no heavy or sticky feel. In some embodiments, the cosmetic composition may be long lasting. For example, the cosmetic composition may stay on a keratinous surface or substrate for at least 4 hours or at least 6 hours or at least 8 hours or at least 10 hours or at least 12 hours or at least 14 hours or at least 16 hours or at least 18 hours or at least 20 hours without interruption of esthetical appearance. In some embodiments, the cosmetic composition may provide fresh/cooling water effect.

### Aqueous Phase Ingredients

### Water

In some embodiments, the cosmetic composition may contain at least 60 mass % or at least 62 mass % or at least 64 mass % or at least 66 mass % or at least 68 mass % of water. In some embodiments the cosmetic composition may contain from about 60 mass % to about 90 mass % or from about 70 mass % to about 80 mass % as per total mass (weight) of the composition. In some embodiments, the cosmetic composition may contain from 65 mass % to 75 mass % or from 67 mass % to 73 mass % or from 68 mass % to 72 mass % or from 69 mass % to 71 mass % of water.

In some embodiments, water may be water that contains an extract dissolved in it. For example, in some embodiments, water may be ginseng water. Please prove additional details.

Ginseng water is a nutrient-rich product made from the root of the ginseng plant, that water is known for its ability to hydrate the skin, provide anti-aging benefits, and deliver antioxidants.

Not bounded by theory, ginseng water is designed to penetrate deeply into the skin, delivering nutrients and antioxidants. It can help to plump and firm the skin, reduce the appearance of fine lines and wrinkles. Additionally, ginseng water can improve the overall health of the skin, providing a natural radiance and glow.

In some embodiments, the composition may include PANAX GINSENG ROOT WATER & BUTYLENE GLYCOL & PHENOXYETHANOL & CHLORPHENESIN (Trade Name: GINSENG WATER-CP) from The Garden of Natural solution Co., Ltd.

### Superabsorbent Polymer

Cosmetic compositions, which contain a superabsorbent polymer, are disclosed, for example, in the French Patent No. FR 2,989,884; U.S. patent application publications Nos. US20100330018; US-2015/0342864; US-2014/0093461; US-2016/0081895; US-2018/0221256; US-2015/0023895; US-2015/0224046; US-2022/0387266; U.S. Pat. No. 9,549,891; and PCT publication No. WO-2021/130677.

The at least one superabsorbent polymer may be in the aqueous phase of the cosmetic composition.

In some embodiments, the at least one superabsorbent polymer may include, for example, but is not limited to, at least one of superabsorbent polymers disclosed in U.S. Patent Application Publication No. US-2018/0221256, which is incorporated herein by reference in its entirety. As used herein, the term “superabsorbent polymer” may refer to a polymer, which can absorb and retain an amount of liquid, such as water, which has a mass significantly higher than the mass of the polymer itself. For example, the superabsorbent polymer can absorb and retain an amount of liquid, such as water, which has a mass at least 3 times, or at least 5 times or at least 8 times or at least 12 times or at least 15 times or at least 18 times or at least 20 times than a mass of the polymer itself.

In some embodiments, the at least one superabsorbent polymer may include at least one of cross-linked sodium acrylates, acrylate grafted starches or mixtures thereof.

In some embodiments, the at least one superabsorbent polymer may include, but is not limited to, at least one superabsorbent polymer selected from the group consisting of sodium polyacrylate, sodium acrylate, sodium polyacrylate starch, sodium acrylates crosspolymer-2 or mixtures thereof. For example, in certain embodiments, the at least one superabsorbent polymer may comprise sodium acrylates crosspolymer-2. Sodium acrylates crosspolymer-2 is commercially available, for example, as Barcril AV or Aqua Keep 10SH-NFC from Kobo.

An amount of the at least one superabsorbent polymer in the composition may vary. For example, the composition may contain the superabsorbent polymer, such as sodium acrylates crosspolymer-2 in the amount from about 0.1 mass % to about 3 mass % or from about 0.1 mass % to about 1.5 mass % or from 0.5 mass % to about 1.5 mass % or from about 0.2 mass % to about 1 mass % or from about 0.3 mass % to about 0.7 mass %.

The superabsorbent polymer, such as sodium acrylates crosspolymer-2, may provide the cosmetic composition with a unique change in texture during the application of the composition to a keratinous surface, such as a skin, filling in fine lines, blurring and living a soft powdery finish. The superabsorbent polymer, such as sodium acrylates crosspolymer-2, may have benefits for skin such as smoothening and softening. The superabsorbent polymer, such as sodium acrylates crosspolymer-2, may form a film or a barrier on the skin to result in a smooth feel. The superabsorbent polymer, such as sodium acrylates crosspolymer-2, may act as a thickening agent for improving the texture of formulations.

### Polymer Thickener

In some embodiments, the composition may comprise at least one polymer thickener, such as at least one non-sugar polymer thickener. The at least one polymer thickener, such as at least one non-sugar polymer thickener may be in the aqueous phase of the cosmetic composition. In some embodiments, the at least one non-sugar polymer thickener may include at least one taurate based polymer thickener. In some embodiments, the at least one non-sugar polymer thickener may include at least two taurate based polymer thickeners.

Non-limiting examples of taurate based polymer thickeners include Ammonium Acryloyldimethyltaurate/VP Copolymer (Aristoflex® AVC) and Hydroxyethyl Acrylate/Sodium Acryloyldimethyl Taurate Copolymer (Sepinov™ EMT 10). Aristoflex® AVC is commercially available from CLARIANT INTERNATIONAL LTD., Sepinov™ EMT 10 is commercially available from SEPPIC.

An amount of the at least one taurate based polymer thickener or at least two taurate based polymer thickeners in the composition may vary. In some embodiments, the composition may contain the at least one taurate based polymer thickener or at least two taurate based polymer thickeners in the amount from 0.3 mass % to 3.5 mass % or from 0.5 mass % to 3 mass % or from 0.7 mass % to 2.5 mass % or from 1.0 mass % to 2.5 mass % or from 1.0 mass to 2.2 mass % or from 1.2 mass % to 2.0 mass % or from 1.4 mass % to 1.8 mass %.

In some embodiments, the composition may include both Ammonium Acryloyldimethyltaurate/VP Copolymer (Aristoflex® AVC) and Hydroxyethyl Acrylate/Sodium Acryloyldimethyl Taurate Copolymer (Sepinov™ EMT 10). In some embodiments, the combination of Ammonium Acryloyldimethyltaurate/VP Copolymer (Aristoflex® AVC) and Hydroxyethyl Acrylate/Sodium Acryloyldimethyl Taurate Copolymer (Sepinov EMT™ 10) may provide the stabilize the cosmetic composition.

In some embodiments, a combined amount of Ammonium Acryloyldimethyltaurate/VP Copolymer (Aristoflex® AVC) and Hydroxyethyl Acrylate/Sodium Acryloyldimethyl Taurate Copolymer (Sepinov TMEMT 10) may be from 0.3 mass % to 3.5 mass % or from 0.5 mass % to 3 mass % or from 0.7 mass % to 2.5 mass % or 1.0 mass % to 2.5 mass % or from 1.0 mass to 2.2 mass % or from 1.2 mass % to 2.0 mass % or from 1.4 mass % to 1.8 mass %.

Combining Ammonium Acryloyldimethyltaurate/VP Copolymer (Aristoflex® AVC) and Hydroxyethyl Acrylate/Sodium Acryloyldimethyl Taurate Copolymer (Sepinov™ EMT 10) may provide the stabilize the cosmetic composition with the superabsorbent polymer, such as sodium acrylates crosspolymer-2 in the amounts provided in this disclosure, including the Examples, may provide the composition with desired viscosity, texture and performance.

In some embodiments, the composition may include Ammonium Acryloyldimethyltaurate/VP Copolymer (Aristoflex®AVC) in the amount from 0.2 mass % to 2 mass % or from 0.2 mass % to 1.5 mass % or from 0.3 mass % to 1.5 mass % or from 0.3 mass % to 1.2 mass % or from 0.4 mass % to 1.2 mass % or from 0.4 mass % to 0.8 mass %.

In some embodiments, the composition may include Hydroxyethyl Acrylate/Sodium Acryloyldimethyl Taurate Copolymer (Sepinov™ EMT 10) in the amount from 0.2 mass % to 2 mass % or from 0.2 mass % to 1.5 mass % or from 0.3 mass % to 1.2 mass % or from 0.5 mass % to 1.8 mass % or from 0.7 mass % to 1.5 mass % or from 0.8 mass % to 1.2 mass %.

In some embodiments, the composition may include (a) Ammonium Acryloyldimethyltaurate/VP Copolymer (Aristoflex® AVC) in the amount from 0.2 mass % to 2 mass % or from 0.2 mass % to 1.5 mass % or from 0.3 mass % to 1.5 mass % or from 0.3 mass % to 1.2 mass % or from 0.4 mass % to 1.2 mass % or from 0.4 mass % to 0.8 mass % and Hydroxyethyl Acrylate/Sodium Acryloyldimethyl Taurate Copolymer (Sepinov™ EMT 10) in the amount from 0.2 mass % to 2 mass % or from 0.5 mass % to 1.8 mass % or from 0.7 mass % to 1.5 mass % or from 0.8 mass % to 1.2 mass %.

### Additional Thickener

In some embodiments, the composition may contain at least one additional thickener. The at least one additional thickener may be selected from, but is not limited, to natural thickeners, synthetic thickeners and their combinations. For example, in some embodiments, the at least one additional thickener may include at least one natural thickener. Yet in some embodiments, the at least one additional thickener may include at least one synthetic thickener. Suitable additional thickeners are disclosed, for example, in U.S. Patent Application Publication No. 2017007512 and U.S. Pat. No. 6,689,345, each of which is incorporated herein in its entirety. Examples of suitable additional thickeners may include, but are not limited, to succinoglycan, xanthan gum, carrageenan, gellan gum, starch, hydroxyalkylcellulose, pullulan, carbomer, sodium silicate, magnesium silicate, bentonite, acrylamide copolymer, sodium acryloyl dimethyl taurine/hydroxyethyl acrylate copolymer, agar, polyurethane, and hydrophobic polyether polyurethane. In some embodiments, the additional thickener may be a sugar thickener, such as xanthan gum.

The amount of the at least one additional thickener, such as a sugar thickener, e.g. xanthan gum, in the cosmetic composition may vary. For example, the at least one additional thickener, such as a sugar thickener, e.g., xanthan gum, may be present at range from about 0.01 mass % to about 0.5 mass % or from about 0.06 mass % to 0.3 mass % or from about 0.1 mass % to about 0.3 mass %, from about 0.15 mass % to about 0.2 mass %, or about 0.2 mass %.

### Water Soluble Emollient

In some embodiments, the at least one water soluble emollient may comprise a microfluidized emollient composition, i.e. a water soluble composition of a microfluidized emollient. A water soluble emollient may be in the aqueous phase of the composition. Microfluidized emollient compositions are disclosed, for example, in U.S. Patent Application Publication No. US 20160081895, which is incorporated herein by reference in its entirety.

The term “microfluidized” may refer to a fluid, particles of which may be geometrically constrained to a small, typically on a sub-millimeter scale, at which capillary penetration may govern mass transport. In some embodiments, the particle size of the microfluidized substance may be from about 0.1 micron to 2 micron or from about 0.2 micron to about 1.5 micron or from about 0.5 micron to about 1 micron.

The term “emollient” may refer to a substance having ability of softening and smoothing a keratinous surface or substrate, such as skin and hair.

In some embodiments, the at least one water soluble emollient may include microfluidized squalane.

In some embodiments, the microfluidized emollient composition may include an emollient substance, a solvent, and a stabilizing agent.

In some embodiments, the emollient substance of the microfluidized emollient composition may be selected from but not limited to hydrocarbons, including hydrocarbons of natural origin, including saturated branched hydrocarbons. Non-limiting examples of the emollient of the microfluidized emollient composition may be squalane, which may be produced, for example, from an animal source, such as shark liver oil, a vegetable oil, such as an olive oil or a jojoba oil, or a mixture thereof, or an oil, such as a plant based oil, such as a jojoba oil.

In some embodiments, the solvent of the microfluidized emollient composition may be selected from water, humectants and mixtures thereof.

In some embodiments, the stabilizing agent of the microfluidized emollient composition may be selected from surfactants, including but not being limited to triglycerides, fatty acids and carbohydrates and mixtures thereof. A non-limiting example of a stabilizing agent utilized may be hydrogenated lecithin.

In some embodiments, the water soluble microfluidized emollient composition may comprise glycerin, which may be vegetable glycerin; water; squalane, which may be microfluidized squalane; hydrogenated lecithin; or a mixture thereof.

A non-limiting example of water soluble microfluidized emollient composition utilized in the composition is the emollient composition containing water, (vegetable) glycerin, squalane and hydrogenated lecithin. Such a composition is commercially available as Dermanet Squalane from Blue Sun International, Miami, Florida, U.S.A. Dermanet Squalane contains 66% Glycerin, 21.5% Water, 10% Squalane, 2.5% Hydrogenated Lecithin.

A content of the water soluble microfluidized emollient composition, such as Dermanet™ Squalane, in the cosmetic composition may vary. In some embodiments, the water soluble microfluidized emollient composition, such as Dermanet™ Squalane, may be present in the cosmetic composition in the amount from about 1 mass % to about 8 mass % or from about 1 mass % to about 5 mass % or from about 2 mass % to about 5 mass % or from about 2 mass % to about 4 mass % or from about 2.5 mass % to about 3.5 mass %.

Adding the water soluble microfluidized emollient composition, such as Dermanet Squalane, may provide the cosmetic composition with a silky elegant velvety texture, like texture of silicone containing cosmetic compositions.

In some embodiments, the at least one water soluble emollient may be a hydrophilic emollient containing at least one butter. For example, in some embodiments, the hydrophilic emollient may include squalene, a mango seed butter and Orbignya oleifera seed oil.

One non-limiting example of a hydrophilic emollient containing at least one butter may be HEDI® MANGO BUTTER R10076 commercially available from Biocogent LLC. HEDI® MANGO BUTTER R10076 includes WATER & MANGIFERA INDICA (MANGO) SEED BUTTER & ORBIGNYA OLEIFERA SEED OIL & SQUALANE & PHOSPHOLIPIDS & TOCOPHERYL ACETATE & GLUCONOLACTONE & BENZYL ALCOHOL & SODIUM BENZOATE. HEDI® MANGO BUTTER R10076 contains 65% WATER & 15% MANGIFERA INDICA (MANGO) SEED BUTTER &7.5% ORBIGNYA OLEIFERA SEED OIL & 7.5% SQUALANE & 2.0% PHOSPHOLIPIDS & 1.125% GLUCONOLACTONE & 1% TOCOPHERYL 0.5% ACETATE & BENZYL ALCOHOL & 0.375% SODIUM BENZOATE.

A content of the hydrophilic emollient containing at least one butter, such as HEDI® MANGO BUTTER R10076, in the cosmetic composition may vary. In some embodiment, the hydrophilic emollient containing at least one butter, such as HEDI® MANGO BUTTER R10076, may be present in the cosmetic composition in the amount from 0.1 mass % to 3.0 mass % or from 0.1 mass % to 2.5 mass % or from 0.3 mass % to 2.0 mass % or from 0.5 mass % to 2.0 mass % or from 1.5 mass % to 2.5 mass % or from 1.8 mass %. to 2.2 mass %.

In some embodiments, the cosmetic composition may include (a) a water soluble microfluidized emollient composition, such as Dermanet Squalane, in the amount from about 1 mass % to about 8 mass % or from about 1 mass % to about 5 mass % or from about 2 mass % to about 5 mass % or from about 2 mass % to about 4 mass % or from about 2.5 mass % to about 3.5 mass % and (b) a hydrophilic emollient containing at least one butter, such as HEDI® MANGO BUTTER R10076, in the amount from 0.1 mass % to 3.0 mass % or from 0.1 mass % to 2.5 mass % or from 0.3 mass % to 2.0 mass % or from 0.5 mass % to 2.0 mass % or from 1.5 mass % to 2.5 mass % or from 1.8 mass %. to 2.2 mass %.

Use of the two water soluble emollients, such as Dermanet Squalane and HEDI® MANGO BUTTER R10076, in the amounts disclosed in this disclosure, including the Examples, may provide the cosmetic composition with smooth silky and soft cushiony texture.

### Film Former

In some embodiments, the cosmetic composition may include at least one film former. In some embodiments, a film former may be aqueous polyurethane dispersion. A film former may be in the aqueous phase of the composition.

One non-limiting example of a film former may be BAYCUSAN® C 1004/1 (57.5% WATER, 41% POLYURETHANE-35, 1.2% METHYLPROPANEDIOL, 0.25% CAPRYLYL GLYCOL, PHENYLPROPANOL). Baycusan C 1004/1 is a colloidal system of a high molecular weight polyurethane polymer dispersed in water. An internal emulsifier is incorporated into the polymer backbone to ensure stability of the dispersion. Baycusan C 1004/1 may work as a universal film former for high and low viscosity systems. The polymer structure contains both hydrophilic and hydrophobic segments, which impart a unique combination of water resistance and ease of removal from skin

The content of the at least one film former in the cosmetic composition may vary. In some embodiments, the at least one film former may be present in the cosmetic composition in the amount from 2 mass % to 8 mass % or from 2 mass % to 6 mass % or from 2 mass % to 5 mass % or from 2.5 mass % to 5.5 mass % or from 3 mass % to 5 mass % or from 3.5 mass % to 4.5 mass %.

### Oil Phase Ingredients

### Hydrophobic Emollient

In some embodiments, the cosmetic composition may contain at least one hydrophobic emollient. A hydrophobic emollient may be an oil soluble emollient. A hydrophobic emollient may be in an oil phase of the composition.

Non-limiting examples of hydrophobic emollients include: NEOLIGHT 100P (100% ISODECYL NEOPENTANOATE); VEGELIGHT C912-LC (94% C9-12 Alkane, 6% Coco-Caprylate/Caprate); ELDEW PS-203R (99.99% PHYTOSTERYL/OCTYLDODECYL LAUROYL GLUTAMATE & 0.01% TOCOPHEROL; NEOSSANCE™ SQUALANE (SQUALANE).

The content of the at least one hydrophobic emollient in the cosmetic composition may vary. In some embodiments, the at least one hydrophobic emollient may be present in the cosmetic composition in the amount from 3 mass % to 20 mass % or from 3 mass % to 15 mass % or from 4 mass % to 18 mass % or from 5 mass % to 15 mass % or from 7 mass % to 13 mass % or from 10 mass % to 12 mass %.

### Additional Ingredients

In some embodiments, the cosmetic composition may also include additional ingredient(s), such as humectant(s), e.g. glycerin, propanediol and trehalose; preservative(s), e.g. phenoxyethanol; chelating agent(s), e.g. trisodium ethylenediaminetetraacetic acid (EDTA); buffer(s), e.g. sodium titrate and citric acid. A total content of the additional ingredient(s) may be from 1 mass % to 25 mass % or from 10 mass % to 20 mass % or from 1 mass % to 15 mass % or from 2 mass % to 13 mass % or from 3 mass % to 10 mass %.

### Exemplary Compositions

One exemplary cosmetic composition may contain (a) from about 0.3 mass % to about 0.7 mass % of sodium acrylates crosspolymer-2; (b) from about 0.5 mass % to about 1.5 mass % of a hydrophilic emollient comprising squalene, a mango seed butter and Orbignya oleifera seed oil; (c) from about 2.5 mass % to about 3.5 mass % of a water soluble emollient composition comprising glycerin, water, squalane, and hydrogenated lecithin; (d) from 0.4 mass % to 0.8 mass % of Ammonium Acryloyldimethyltaurate/VP Copolymer; and (e) from 0.8 mass % to 1.2 mass % of Hydroxyethyl Acrylate/Sodium Acryloyldimethyl Taurate Copolymer.

### Methods of Making

In some embodiments, the composition may be prepared by mixing ingredients of the water phase in a first container, mixing ingredients of the oil phase in a second container and then mixing the ingredients of both the water phase and the oil phase in a single container.

In some embodiments, the composition may be prepared by mixing some of ingredients of the water in a first container, mixing ingredients of the oil phase in a second container, then mixing the mixed ingredients of the water phase and the mixed ingredients of the oil phase in a single container and then adding additional ingredient(s) of the water phase to the mix.

For example, in some embodiments, water may be mixed with water soluble emollient(s) and a first polymer thickener, such as Ammonium Acryloyldimethyltaurate/VP Copolymer (Aristoflex® AVC), in a first container; while the oil phase ingredients, such as oily (hydrophobic) emollients may be mixed in a second container. Then the contents of the first and the second container may be mixed in a single container. Then, additional ingredients of the water phase, such as a superabsorbent polymer and a second polymer thickener, such as Hydroxyethyl Acrylate/Sodium Acryloyldimethyl Taurate Copolymer (Sepinov™ EMT 10) Ammonium Acryloyldimethyltaurate/VP Copolymer (Aristoflex® AVC) or their combination, may be added to the mix. The resulting mix may be stirred until the second polymer thickener is dispersed.

In a certain embodiment, water may be mixed with a chelating agent and/or buffer in the first container. Then a preservative, such as phenoxyethanol, which may be premixed with a sugar thickener, such as Xanthan Gum, and a humectant, such as glycerin may be added to the first container. Then water soluble emollient(s) may be added to the first container. Then a film former may be added to the first container. Then a first polymer thickener, such as Ammonium Acryloyldimethyltaurate/VP Copolymer (Aristoflex® AVC) may be added to the first container and mixed well until the first polymer thickener is dispersed. Then mixed in a separate container oil phase ingredients, such as oily (hydrophobic) emollient(s), may be added to the first container. Then a superabsorbent polymer, such as Barcril AV, may be added to the first container. Then a second polymer thickener, such as Hydroxyethyl Acrylate/Sodium Acryloyldimethyl Taurate Copolymer (Sepinov™ EMT 10) Ammonium Acryloyldimethyltaurate/VP Copolymer (Aristoflex® AVC) or their combination, may be added to the first container and mixed well until the second polymer thickener is dispersed.

Embodiments described herein are further illustrated by, though in no way limited to, the following working examples.

### Example 1

Two different polymers, Artistoflex AVC and Sepinov EMT 10, were used in the same formulation base with different concentrations to evaluate the performance of the polymers in silicone-free formulations. The compositions of the formulations is shown in Table 1.

TABLE 1
Compositions of Primer Formulations with Different Polymers

| | | | FL-Base | FL #1 | FL #2 | FL #3 | FL #4 |
|---|---|---|---|---|---|---|---|
| Function | RM Name | US INCI | RM % | RM % | RM % | RM % | RM % |
| Humectant | DEIONIZED | WATER | 70.86 | 69.27 | 69.27 | 69.27 | 69.27 |
| | WATER | | | | | | |
| | GLYCERINE | GLYCERIN | 3.00 | 3.00 | 3.00 | 3.00 | 3.00 |
| | NAT 99.7% | | | | | | |
| | ZEMEA | PROPANEDIOL | 5.00 | 5.00 | 5.00 | 5.00 | 5.00 |
| | TREHALOSE | TREHALOSE | 1.00 | 1.00 | 1.00 | 1.00 | 1.00 |
| Preservative | PHENOXETOL NF | PHENOXYETHANOL | 0.90 | 0.90 | 0.90 | 0.90 | 0.90 |
| Chelating agent | EDTA 3•NA2•H20 | TRISODIUM EDTA | 0.02 | 0.02 | 0.02 | 0.02 | 0.02 |
| Buffer | SODIUM CITRATE | SODIUM CITRATE | 0.01 | 0.01 | 0.01 | 0.01 | 0.01 |
| | CITRIC ACID | CITRIC ACID | 0.01 | 0.01 | 0.01 | 0.01 | 0.01 |
| Additional | SILIGEL ™ | XANTHANGUM&SCLEROTIUMGUM&LEC- | 0.20 | 0.20 | 0.20 | 0.20 | 0.20 |
| (sugar) | | ITHIN&PULLULAN& SILICA | | | | | |
| thickener | | | | | | | |
| Water soluble | HEDI | WATER & MANGIFERA INDICA (MANGO) | 1.00 | 1.00 | 1.00 | 1.00 | 1.00 |
| (hydrophilic)Emol- | MANGO | SEED BUTTER & ORBIGNYA OLEIFERA | | | | | |
| lients | BUTTER | SEED OIL & SQUALANE & | | | | | |
| | R10076 | PHOSPHOLIPIDS & TOCOPHERYL | | | | | |
| | | ACETATE & GLUCONOLACTONE & | | | | | |
| | | BENZYL ALCOHOL & SODIUM BENZOATE | | | | | |
| | DERMANET | GLYCERIN & WATER & SQUALANE & | 3.00 | 3.00 | 3.00 | 3.00 | 3.00 |
| | SQUALANE | HYDROGENATED LECITHIN | | | | | |
| Film Former | BAYCUSAN ® | WATER, POLYURETHANE-35, | 4.00 | 4.00 | 4.00 | 4.00 | 4.00 |
| | C1004/1 | METHYLPROPANEDIOL, CAPRYLYL | | | | | |
| | | GLYCOL, PHENYLPROPANOL | | | | | |
| Oily | NEOLIGHT | ISODECYL NEOPENTANOATE | 4.00 | 4.00 | 4.00 | 4.00 | 4.00 |
| (hydrophobic) | 100P | | | | | | |
| emollient | VEGELIGHT | C9-12 Alkane, | 2.00 | 2.00 | 2.00 | 2.00 | 2.00 |
| | C912-LC | Coco-Caprylate/Caprate | | | | | |
| | ELDEW | PHYTOSTERYL/OCTYLDODECYL | 1.00 | 1.00 | 1.00 | 1.00 | 1.00 |
| | PS-203R | LAUROYL GLUTAMATE & TOCOPHEROL | | | | | |
| | NEOSSANCE ™ | SQUALANE | 4.00 | 4.00 | 4.00 | 4.00 | 4.00 |
| | SQUALANE | | | | | | |
| superabsorbent | BARCRIL | SODIUM ACRYLATES CROSSPOLYMER-2 | — | — | — | — | — |
| polymer. | AV | | | | | | |
| polymer (non- | ARISTOFLEX | AMMONIUM | — | 1.60 | — | 0.60 | 1.00 |
| sugar) | AVC | ACRYLOYLDIMETHYLTAURATE/VP | | | | | |
| thickeners | | COPOLYMER | | | | | |
| | Sepinov | Hydroxyethyl Acrylate/Sodium | — | | 1.60 | 1.00 | 0.60 |
| | EMT 10 | Acryloyldimethyl Taurate Copolymer | | | | | |
| | | Total | 100.00 | 100.00 | 100.00 | 100.00 | 100.00 |

The compositions of Table 1 were prepared using the following procedure:
1. Water, Chelating agent, and buffer were combined in a main beaker. Mixing started.
2. Glycerin with Siligel and Phenoxyethanol were premixed, then added to the main beaker.
3. Mango butter and Dermanet squalene were added and the mixing continued.
4. Baycusan ® C was added and mixed well.
5. Aristoflex ® AVC was added and mixed until dispersed. A batch was thicken.
6. Oily emollients were combined on a side beaker, then added to the main beaker.
7. Zemea with Barcril-AV were pre-mixed, then added to the main beaker. Mix well
8. Polymer thickeners, such as Sepinov EMT 10 ™, Aristoflex ® AVC, or their mix, were added to the main beaker, mixed well until dispersed. The batch was dropped into a storage container.

In order to evaluate the effect of the polymers on the primer performance, a standard test formulation (FL-base) was used. Two polymer thickeners, Aristoflex AVC and Sepinov EMT 10, were used in the initial evaluations (FL #1, 2, 3, 4). The effects of both polymers on the primer performance were evaluated by varying their concentration. The viscosity, pH, and texture of the four formulations are summarized in table 2. Viscosity was measured by Brookfield Ametek DV-I viscometer, Spindle RV #TE, share rate is 10 rpm. pH was measured by Thermo Scientific (Orion Star A 215) pH Conductivity Meter. Texture was evaluated by inventors, by applying FL samples on a face.

TABLE 2
Viscosity, pH, and Texture Comparison

| | Formula# | | | | |
|---|---|---|---|---|---|
| | FL- Base | FL# 1 | FL# 2 | FL# 3 | FL# 4 |
| | W/W % | | | | |
| | % | % | % | % | % |
| Aristoflex AVC | 0 | 1.6 | 0 | 0.6 | 1 |
| Sepinov EMT 10 | 0 | 0 | 1.6 | 1 | 0.6 |
| Barcril AV | 0 | 0 | 0 | 0 | 0 |
| pH | | 5.71 | 5.93 | 5.79 | 5.51 |
| Viscosity, Pa · S | | 33,080 | 25,000 | 28,500 | 33,250 |
| Texture | | Light-soft gel | Light-Soft gel | Soft-smooth | Soft-smooth |
| | | cream | Cream | light gel cream | longer playtime |
| Stability | Unstable | Stable | Stable | Stable | Stable |

The data in Table 2 shows the following:
(1) FL# 1 and 2 stabilized the FL-base by adding a single polymer. They gave the look & feel of typical Silicone-Free cream, without the silky and silicone-like texture
(2) FL# 3 and 4 improved texture by adding combinations of the 2 polymers with the same total concentration (1.6%).
(3) FL# 4 lacks quick-absorption character desired for a primer, i.e. has a longer play time.
(4) FL# 3 presents a better (shorter) play time as a primer.

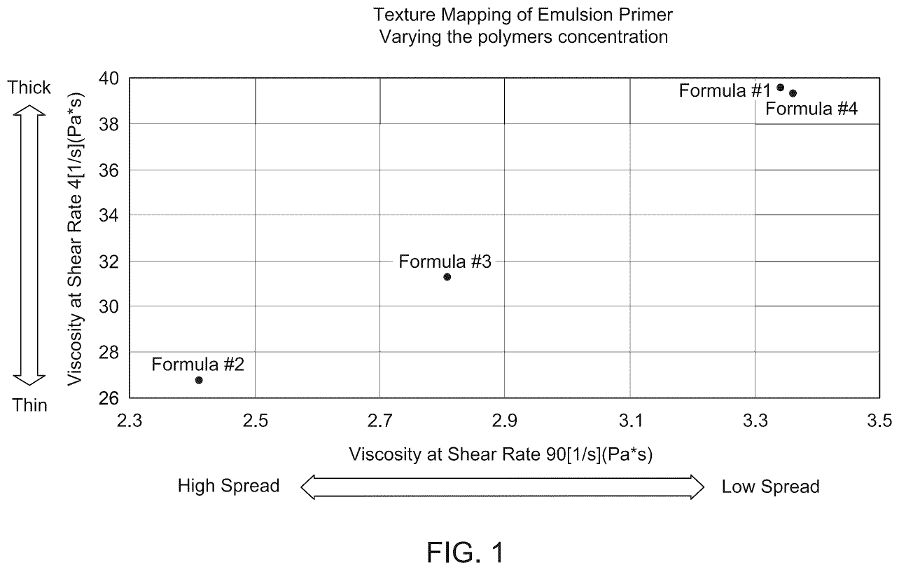

Characterization of the rheological properties of FL #1,2,3, and #4 using Discovery HR 20 Rheometer by TA instruments Texture mapping of the four formulations is shown in FIG. 1. The data in FIG. 1 demonstrates that FL #3 shows the better spread ability and perfect playtime for a primer. A more effective ratio of Artistoflex AVC to Sepinov EMT 10 may be 0.6% to 1.0%.

### Example 2

FL #3 in Example 1 resulted in a better texture by combining the two polymers (Artistoflex AVC and Sepinov EMT 10) with a certain ratio. However, FL #3 in Example 1 lacked a silicone-like feel. In order to improve the texture, super absorbent polymer Barcril AV was added into the FL #3 with different concentrations (FL #5 and 6). The composition of the formulations is shown in Table 3.

TABLE 3
Compositions of Primer Formulations with Different Polymers

| | | | FL #3 | FL# 5 | FL #6 |
|---|---|---|---|---|---|
| Function | RM Name | US INCI | RM % | RM % | RM % |
| Humectant | DEIONIZED | WATER | 69.27 | 68.77 | 68.27 |
| | WATER | | | | |
| | GLYCERINE NAT | GLYCERIN | 3.00 | 3.00 | 3.00 |
| | 99.7% | | | | |
| | ZEMEA | PROPANEDIOL | 5.00 | 5.00 | 5.00 |
| | TREHALOSE | TREHALOSE | 1.00 | 1.00 | 1.00 |
| Preservative | PHENOXETOL NF | PHENOXYETHANOL | 0.90 | 0.90 | 0.90 |
| Chelating agent | EDTA 3.NA2.H20 | TRISODIUM EDTA | 0.02 | 0.02 | 0.02 |
| Buffer | SODIUM | SODIUM CITRATE | 0.01 | 0.01 | 0.01 |
| | CITRATE | | | | |
| | CITRIC ACID | CITRIC ACID | 0.01 | 0.01 | 0.01 |
| Additional | SILIGEL ™ | XANTHANGUM&SCLEROTIUM- | 0.20 | 0.20 | 0.20 |
| (Sugar) thickener | | GUM&LECITHIN&PULLULAN& | | | |
| | | SILICA | | | |
| Water soluble | HEDI MANGO | WATER & MANGIFERA INDICA | 1.00 | 1.00 | 1.00 |
| (hydrophilic) | BUTTER R10076 | (MANGO) SEED BUTTER & | | | |
| Emollients | | ORBIGNYA OLEIFERA SEED | | | |
| | | OIL & SQUALANE & | | | |
| | | PHOSPHOLIPIDS & TOCOPHERYL | | | |
| | | ACETATE & GLUCONOLACTONE & | | | |
| | | BENZYL ALCOHOL & SODIUM | | | |
| | | BENZOATE | | | |
| | DERMANET | GLYCERIN & WATER & | 3.00 | 3.00 | 3.00 |
| | SQUALANE | SQUALANE & HYDROGENATED | | | |
| | | LECITHIN | | | |
| Film Former | BAYCUSAN ® C | WATER, POLYURETHANE-35, | 4.00 | 4.00 | 4.00 |
| | 1004/1 | METHYLPROPANEDIOL, | | | |
| | | CAPRYLYL GLYCOL, | | | |
| | | PHENYLPROPANOL | | | |
| Oily | NEOLIGHT 100P | ISODECYL NEOPENTANOATE | 4.00 | 4.00 | 4.00 |
| (hydrophobic) | VEGELIGHT | C9-12 Alkane, Coco-Caprylate/Caprate | 2.00 | 2.00 | 2.00 |
| Emollients | C912-LC | | | | |
| | ELDEW PS-203R | PHYTOSTERYL/OCTYLDODECYL | 1.00 | 1.00 | 1.00 |
| | | LAUROYL GLUTAMATE & | | | |
| | | TOCOPHEROL | | | |
| | NEOSSANCE ™ | SQUALANE | 4.00 | 4.00 | 4.00 |
| | SQUALANE | | | | |
| superabsorbent | BARCRIL AV | SODIUM ACRYLATES | — | 0.50 | 1.00 |
| polymer | | CROSSPOLYMER-2 | | | |
| polymer | ARISTOFLEX | AMMONIUM ACRYLOYLDIMETHYL- | 0.60 | 0.60 | 0.60 |
| thickeners | AVC | TAURATE/VP COPOLYMER | | | |
| | Sepinov EMT 10 | Hydroxyethyl Acrylate/Sodium Acryloyl- | 1.00 | 1.00 | 1.00 |
| | | dimethyl Taurate Copolymer | | | |
| | | Total | 100.00 | 100.00 | 100.00 |

The compositions of Table 3 were prepared using the same methods as the compositions of Table 1.

To improve the texture of FL #3 for Silky-Smooth feel to match silicone-based formulation, another polymer was added to the formulation, and performance evaluation of the formulations are summarized in Table 4. The viscosity, pH and texture evaluations were performed using the same methods as in Table 2.

TABLE 4
Viscosity, pH, and Texture Comparison

| | Formula# | | |
|---|---|---|---|
| | FL# 3 | FL# 5 | FL# 6 |
| | W/W % | | |
| | % | % | % |
| Aristoflex AVC | 0.6 | 0.6 | 0.6 |
| Sepinov EMT 10 | 1 | 1 | 1 |
| Barcril AV | 0 | 0.5 | 1.0 |
| pH | 5.79 | 5.84 | 5.45 |
| Viscosity | 28,500 | 35,670 | 74,860 |
| Texture | Soft-smooth | Light-touch | A little |
| | light gel | excellent | heavy-touch |
| | cream | smooth; soft | Smooth, |
| | | cushioning | blurring |
| | | and blurring | like Silicone |
| | | effect | Elastomer |
| Stability | Stable | Stable | Stable |

The data in Table 4 shows the following:
(1) FL# 5 and 6 improved texture in Silky-smooth silicone feel.
(2) The viscosity of the FL#6 is high. The texture is a little heavy.
(3) FL#5 may have a ratio of the three polymers which provided the preferred light texture, viscosity, and smooth application for a primer.

### Example 3

To improve the Silky-Rich feel of the formulation, two Hydrophilic Emollients was tested separately, as well as combined together. The composition of the formulations are listed in Table 5, along with the control FL #10.

TABLE 5
The Effect of Hydrophilic Emollients on Primer Texture

| | | | FL# 5 | FL #8 | FL #9 | FL #10 |
|---|---|---|---|---|---|---|
| Function | RM Name | US INCI | RM % | RM % | RM % | RM % |
| Humectant | DEIONIZED | WATER | 68.76 | 71.76 | 69.76 | 72.76 |
| | WATER | | | | | |
| | GLYCERINE | GLYCERIN | 3.00 | 3.00 | 3.00 | 3.00 |
| | NAT 99.7% | | | | | |
| | ZEMEA | PROPANEDIOL | 5.00 | 5.00 | 5.00 | 5.00 |
| | TREHALOSE | TREHALOSE | 1.00 | 1.00 | 1.00 | 1.00 |
| Preservative | PHENOXETOL NF | PHENOXYETHANOL | 0.90 | 0.90 | 0.90 | 0.90 |
| | 272556 | | | | | |
| chelating | EDTA 3•NA2•H20 | TRISODIUM EDTA | 0.02 | 0.02 | 0.02 | 0.02 |
| agent | | | | | | |
| Buffer | SODIUM CITRATE | SODIUM CITRATE | 0.01 | 0.01 | 0.01 | 0.01 |
| | CITRIC ACID | CITRIC ACID | 0.01 | 0.01 | 0.01 | 0.01 |
| | ANHYDROUS | | | | | |
| Additional | SILIGEL ™ | XANTHAN GUM & SCLEROTIUM GUM & | 0.20 | 0.20 | 0.20 | 0.20 |
| (sugar) | | LECITHIN & PULLULAN & SILICA | | | | |
| thickener | | | | | | |
| Water soluble | HEDI MANGO | WATER & MANGIFERA INDICA (MANGO) SEED BUTTER | 1.00 | 1.00 | — | — |
| (hydrophilic) | BUTTER | & ORBIGNYA OLEIFERA SEED OIL & SQUALANE & | | | | |
| Emollients | R10076 | PHOSPHOLIPIDS & TOCOPHERYL ACETATE & | | | | |
| | | GLUCONOLACTONE & BENZYL ALCOHOL & | | | | |
| | | SODIUM BENZOATE | | | | |
| | DERMANET | GLYCERIN & WATER & SQUALANE & | 3.00 | — | 3.00 | — |
| | SQUALANE | HYDROGENATED LECITHIN | | | | |
| Film Former | BAYCUSAN ® | WATER, POLYURETHANE-35, METHYLPROPANEDIOL, | 4.00 | 4.00 | 4.00 | 4.00 |
| | C1004/1 | CAPRYLYL GLYCOL, PHENYLPROPANOL | | | | |
| Oily | NEOLIGHT 100P | ISODECYL NEOPENTANOATE | 4.00 | 4.00 | 4.00 | 4.00 |
| (hydrophobic) | VEGELIGHT | C9-12 Alkane, Coco-Caprylate/Caprate | 2.00 | 2.00 | 2.00 | 2.00 |
| Emollients | C912-LC | | | | | |
| | ELDEW | PHYTOSTERYL/OCTYLDODECYL LAUROYL | 1.00 | 1.00 | 1.00 | 1.00 |
| | PS-203R | GLUTAMATE & TOCOPHEROL | | | | |
| | NEOSSANCE ™ | | 4.00 | 4.00 | 4.00 | 4.00 |
| | SQUALANE | | | | | |
| superabsorbent | BARCRIL | SODIUM ACRYLATES CROSSPOLYMER-2 | 0.50 | 0.50 | 0.50 | 0.50 |
| polymer | AV | | | | | |
| Polymers | ARISTOFLEX | AMMONIUM ACRYLOYLDIMETHYLTAURATE/VP | 0.60 | 0.60 | 0.60 | 0.60 |
| (non-sugar) | AVC | COPOLYMER | | | | |
| thickener | Sepinov | Hydroxyethyl Acrylate/Sodium | 1.00 | 1.00 | 1.00 | 1.00 |
| | EMT 10 | Acryloyldimethyl Taurate Copolymer | | | | |
| | | Total | 100.00 | 100.00 | 100.00 | 100.00 |

The compositions of Table 5 were prepared using the same methods as the compositions of Table 1.

Table 6 provides results of evaluation of an effect of hydrophilic emollients on texture of the formulations from Table 5. The viscosity, pH and texture evaluations were performed using the same methods as in Table 2.

TABLE 6
Viscosity, pH, and Texture Comparison

| | FL# 5 | FL# 8 | FL# 9 | FL# 10 |
|---|---|---|---|---|
| Trade name | w/w % | w/w % | w/w % | w/w % |
| HEDI MANGO | 1.00 | 1.00 | 0.00 | 0.00 |
| BUTTER R10076 | | | | |
| DERMANET | 3.00 | 0.00 | 3.00 | 0.00 |
| SQUALANE | | | | |
| pH | 5.88 | 5.83 | 5.94 | 5.92 |
| Viscosity | 34,750 | 34,420 | 35,150 | 34,580 |
| Texture | Excellent smooth | Light, fast | Soft-smooth | not silky, |
| | soft cushioning and | drying | light gel cream | missing the |
| | blurring effect | | | velvety finish |
| Stability | Stable | Stable | Stable | Stable |

The data in Table 6 shows that FL #5 using HEDI Mango Butter at 1% and Dermanet Squalane at 3% provides silky elegant texture similar to silicone-containing compositions.

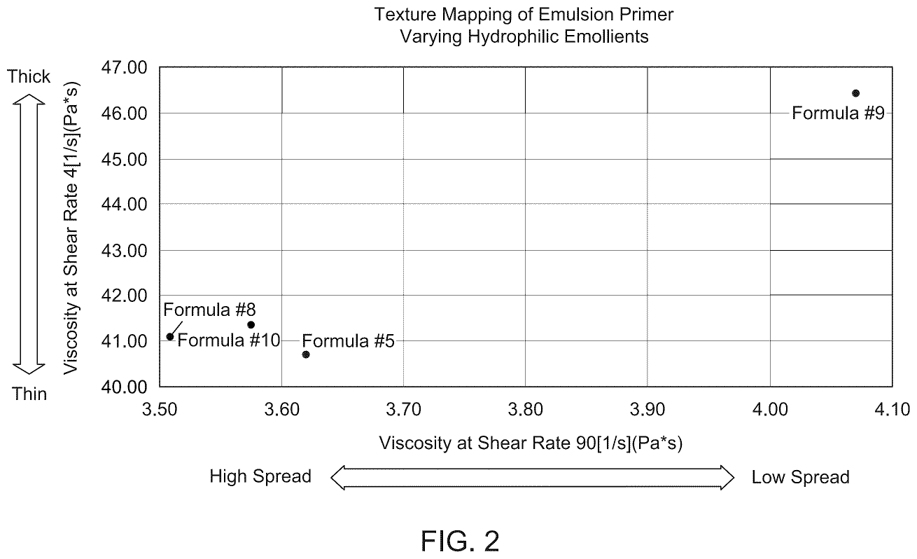

Characterization of the rheological properties of FL #5, 8, 9, and 10 using Discovery HR 20 Rheometer by TA instruments Texture mapping of the four formulations is shown in FIG. 2.

The data in FIG. 2 demonstrates that the formulas without Dermanet Squalane (Formula #8 and #10) absorbed much faster, therefore; not enough playtime to feel the luxurious soft and smooth texture. Adding Dermanet Squalane and Mango Butter (FL #5) provided a silky elegant texture like silicones. Similarly to the data in FIG. 1, the data in FIG. 2 was acquired using TA Discovery HR20 Rheometer.

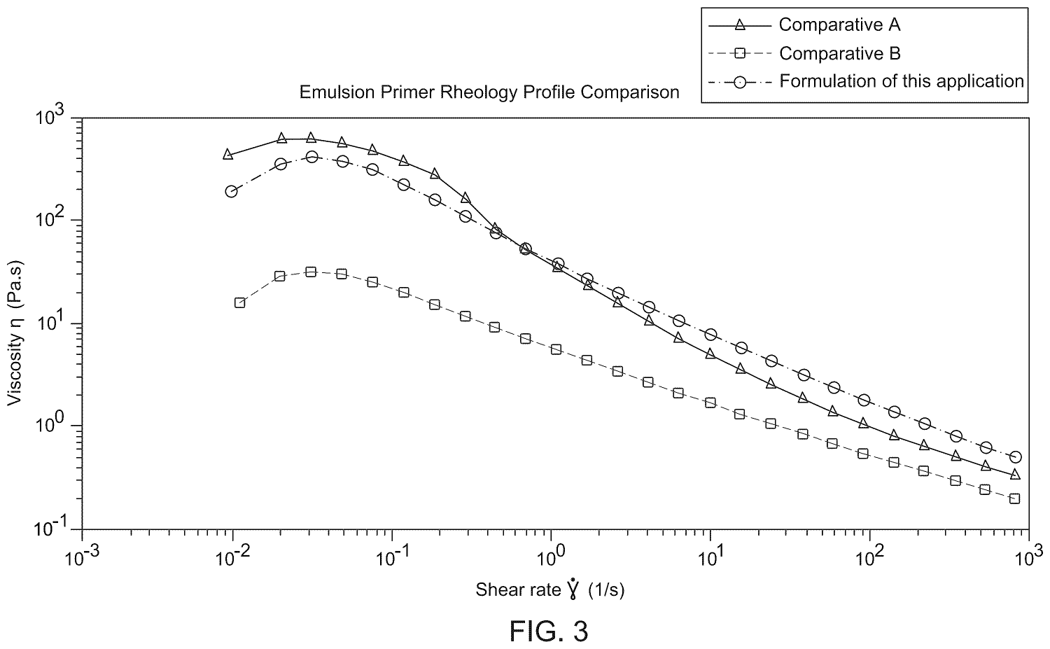

The Composition, which includes all the ingredients of the formulation of formula #5 plus pearls and was compared with benchmark formulations of Silicone-based Primer (Comparative A) (and Silicone-Free Primer (Comparative B) that are commercially available products. The flow profile comparison is shown in FIG. 3. The Shear Rate Range in FIG. 3 is 0.01 to 1000 (1/s).

Comparative A ingredients: (Water\Aqua\Eau, Dimethicone, Butylene Glycol, Cetyl Ricinoleate, Trisiloxane, Glyceryl Stearate Se, Glyceryl Stearate, Peg-100 Stearate, Simmondsia Chinensis (Jojoba) Seed Oil, Butyrospermum Parkii (Shea Butter), Cetyl Esters, Algae (Seaweed) Extract, Cetyl Alcohol, Glycerin, Sesamum Indicum (Sesame) Seed Oil, Medicago Sativa (Alfalfa) Seed Powder, Helianthus Annuus (Sunflower) Seedcake, Prunus Amygdalus Dulcis (Sweet Almond) Seed Meal, Eucalyptus Globulus (Eucalyptus) Leaf Oil, Sodium Gluconate, Copper Gluconate, Calcium Gluconate, Magnesium Gluconate, Zinc Gluconate, Tocopheryl Succinate, Niacin, Sesamum Indicum (Sesame) Seed Powder, Caffeine, Panthenol, Acetyl Glucosamine, Sigesbeckia Orientalis (St. Paul's Wort) Extract, Citrus Aurantifolia (Lime) Peel Extract, Camellia Sinensis (Green Tea) Leaf Extract, Undaria Pinnatifida Extract, Sorbitol, Sodium Hyaluronate, Tourmaline, Porphyridium Cruentum Extract, Pyrus Malus (Apple) Fruit Extract, Cucumis Sativus (Cucumber) Fruit Extract, Asparagopsis Armata Extract, Ascophyllum Nodosum Extract, Ceramide Ng, Scutellaria Baicalensis Root Extract, Tocopheryl Acetate, Hydrolyzed Rice Extract, Acetyl Hexapeptide-8, Laminaria Digitata Extract, Sucrose, Magnesium Ascorbyl Phosphate, Acrylamide/Sodium Acryloyldimethyltaurate Copolymer, Caprylyl Glycol, Yeast Extract\Faex\Extrait De Levure, Isohexadecane, Xanthan Gum, Citric Acid, Malt Extract, Polysorbate 80, Hexylene Glycol, Fragrance (Parfum), Alcohol Denat., Tin Oxide, Silica, Sodium Citrate, Disodium Edta, Bht, Sodium Sulfite, Sodium Metabisulfite, Phenoxyethanol, Benzyl Benzoate, Coumarin, Linalool, Limonene, Citronellol, Hexyl Cinnamal, Amyl Cinnamal, Mica, Titanium Dioxide (Ci 77891), Red 4 (Ci 14700)).

Comparative B ingredients: (Aqua (Water), Alcohol, Glycerin, Squalane, Betaine, Butylene Glycol, Limnanthes Alba (Meadowfoam) Seed Oil, Polyglycerin-3, Lauroyl Lysine, Hibiscus Sabdariffa Flower Extract, Iris Florentina Root Extract, Synthetic Fluorphlogopite, Hydroxyethyl Acrylate/Sodium Acryloyldimethyl Taurate Copolymer, Polyglycerin-6, Caprylyl Glycol, Hydrogenated Lecithin, Pentylene Glycol, Chlorphenesin, Sodium Citrate, Sodium Hyaluronate, Parfum (Fragrance), Alcaligenes Polysaccharides, Polysorbate 60, Sorbitan Isostearate, Sodium Surfactin, Tin Oxide, Hydrolyzed Viola Tricolor Extract, Citric Acid, 1,2-Hexanediol, Sodium Benzoate, Potassium Sorbate, Tocopherol, [+/−Ci 77891 (Titanium Dioxide)])

FIG. 3 shows that the texture of the formulation of formula 5 (silicone free) is comparable and very similar to Comparative A primer (benchmark with silicone).

Table 7 shows viscosity of formulas 1-10 from Tables 1, 3 and 5 at different temperatures. Experiments confirmed that varying the polymers/thickeners result in change in viscosity. All formulas passed 4-week stability. In all the experiments, formulations of formula 1-10 were monitored for 4 weeks stability at multiple temperatures, such as −5° C., 25° C., 37° C., 45° C. and 50° C. pH and viscosity were measured after 4 weeks at various temperatures.

TABLE 7
Stability @ 4 weeks at Multiple Temperature Conditions

| Viscosity | | | | | | | | | |
|---|---|---|---|---|---|---|---|---|---|
| @4 weeks | FL#1 | FL#2 | FL#3 | FL#4 | FL#5 | FL#6 | FL#8 | FL#9 | FL#10 |
| −5° C. | 33,580 | 25,420 | 28,580 | 33,250 | 35,750 | 86,920 | 36,580 | 44,080 | 38,920 |
| 25° C. | 36,580 | 25,170 | 28,830 | 35,330 | 37,750 | 87,360 | 37,500 | 45,670 | 37,670 |
| 37° C. | 36,080 | 23,670 | 27,920 | 34,580 | 35,920 | 84,560 | 36,250 | 41,250 | 38,330 |
| 45° C. | 33,420 | 23,500 | 27,670 | 33,330 | 36,750 | 83,280 | 35,670 | 44,000 | 37,420 |
| 50° C. | 33,830 | 23,920 | 23,920 | 33,670 | 33,250 | 84,120 | 36,080 | 38,580 | 37,750 |

The results in Table 8 show pH of all the formulations is consistent at all the temperature after 4 weeks.

| PH @4 | | | | | | | | | |
|---|---|---|---|---|---|---|---|---|---|
| weeks | FL#1 | FL#2 | FL#3 | FL#4 | FL#5 | FL#6 | FL#8 | FL#9 | FL#10 |
| −5° C. | 5.89 | 5.79 | 5.76 | 5.69 | 5.91 | 5.48 | 5.92 | 5.87 | 5.86 |
| 25° C. | 5.74 | 5.96 | 5.77 | 5.69 | 5.85 | 5.39 | 5.89 | 5.91 | 5.88 |
| 37° C. | 5.78 | 5.96 | 5.85 | 5.80 | 5.91 | 5.44 | 5.88 | 5.91 | 5.89 |
| 45° C. | 5.78 | 5.99 | 5.84 | 5.75 | 5.82 | 5.40 | 5.86 | 5.89 | 5.87 |
| 50° C. | 5.75 | 5.97 | 5.89 | 5.77 | 5.88 | 5.38 | 5.88 | 5.89 | 5.84 |

The efficacy of the Composition Primer, which includes all the ingredients of the formulation of formula #5, has been tested through a consumer test. The details of the testing result were shown in Table 9. As can be seen, the formulation received very positive rating in overall, especially the performance as a primer. Such as easy apply with refreshing (100%), lightweight (98%), not feel greasy (90.3%), creates a smooth canvas for makeup application (92%).

TABLE 9
Consumer Report for the Composition Primer, which includes
all the ingredients of the formulation of formula 5

| | |
|---|---|
| Number of | 62 |
| subjects | |
| Age range | 18-55 |
| Ethnicity | A11 |
| Skin Type | A11 |
| Inclusion criteria | Regular users of primer and liquid foundation. Mild to moderate: skin |
| | roughness and dullness |
| Usage instructions | Apply after usual skincare regimen. Smooth onto face with fingertips. |
| | Allow primer to absorb and set before applying foundation |

| | |
|---|---|
| Consumer Perception Assessment | % Top two responses (Strongly agree & Agree) |

Primer Alone

| | | Immediate | Day 1 | 1 week | 4 weeks |
|---|---|---|---|---|---|
| 1. | The product is easy to apply | 100.00% | N/A | N/A | N/A |
| 2. | The product blends easily and | 100.00% | N/A | N/A | N/A |
| | effortlessly | | | | |
| 3. | The application of the product | 88.71% | N/A | N/A | N/A |
| | feels refreshing, like a burst of | | | | |
| | water on my skin | | | | |
| 4. | The product feels lightweight | 98.39% | N/A | N/A | N/A |
| | on my skin | | | | |
| 5. | The product does not feel | 98.38% | N/A | N/A | N/A |
| | heavy on my skin | | | | |
| 6. | The product does not feel | 90.32% | N/A | N/A | N/A |
| | greasy on my skin | | | | |
| 7. | The product creates a smooth | 91.94% | N/A | N/A | N/A |
| | canvas for makeup | | | | |
| | application | | | | |
| 8. | The product provides a thin, | 96.78% | N/A | N/A | N/A |
| | breathable barrier/veil on my | | | | |
| | skin | | | | |
| 9. | The product adheres to my | 95.16% | N/A | N/A | N/A |
| | skin well | | | | |
| 10. | The product has a second | 88.71% | N/A | N/A | N/A |
| | skin, flexible feel | | | | |
| 11. | The product dries down | 87.10% | N/A | N/A | N/A |
| | quickly after application | | | | |
| 12. | My skin feels instantly | 95.16% | N/A | N/A | N/A |
| | prepped for makeup | | | | |
| | application | | | | |
| 13. | The product feels gentle on | 98.39% | N/A | N/A | N/A |
| | my skin | | | | |
| 14. | My skin looks healthier | 85.49% | 80.64% | 87.09% | 88.71% |
| 15. | The product brings an even | 87.09% | 88.71% | 95.16% | 91.94% |
| | look to my skin | | | | |
| 16. | The product perfects the look | 85.49% | 85.48% | 88.71% | 93.55% |
| | of my skin | | | | |
| 17. | My skin looks younger | 70.97% | 75.80% | 72.58% | 77.42% |
| 18. | My skin looks brighter | 87.10% | 85.48% | 93.55% | 90.32% |
| 19. | The product makes my skin | 90.33% | 90.32% | 100.00% | 100.00% |
| | look smooth | | | | |
| 20. | My skin feels smooth | 95.16% | 96.77% | 96.77% | 98.39% |
| 21. | My skin feels silky | 93.55% | 87.09% | 98.39% | 95.16% |
| 22. | The product feels like a | 88.71% | 93.55% | 90.32% | 90.32% |
| | skincare/makeup hybrid | | | | |
| 23. | The product retexturizes the | N/A | 72.58% | 80.65% | 85.48% |
| | look of my skin all day | | | | |
| 24. | My skin's natural luminosity | N/A | 77.42% | 83.88% | 93.55% |
| | looks enhanced | | | | |
| 25. | My skin appears to have a | N/A | 75.81% | 82.26% | 87.10% |
| | hydrated plumpness | | | | |
| 26. | My skin looks firmer | N/A | 72.58% | 83.87% | 85.48% |
| 27. | My skin looks more supple | N/A | 79.03% | 83.87% | 88.71% |
| 28. | My skin appears to have a lit- | N/A | 77.42% | 80.64% | 79.03% |
| | from-within glow | | | | |
| 29. | The product gives my skin | N/A | 77.42% | 87.10% | 85.49% |
| | with a flawless appearance | | | | |
| 30. | The product works well with | N/A | 96.77% | 100.00% | 96.77% |
| | my skin tone | | | | |
| 31. | The product reduces the look | N/A | 82.26% | 85.49% | 83.87% |
| | of my fine lines and wrinkles | | | | |
| 32. | The product blurs the look of | N/A | 72.59% | 87.10% | 88.71% |
| | my imperfections | | | | |
| 33. | The product minimizes the | N/A | 80.65% | 80.65% | 82.26% |
| | appearance of my pores | | | | |
| 34. | The product neutralizes the | N/A | 79.04% | 85.48% | 85.48% |
| | look of redness | | | | |
| 35. | My skin feels soft | N/A | 95.16% | 100.00% | 100.00% |
| 36. | The product feels comfortable | N/A | 93.55% | 100.00% | 98.39% |
| | to wear all day long | | | | |
| 37. | The product feels hydrating | N/A | 88.71% | 93.55% | 93.55% |
| | on my skin | | | | |
| 38. | My skin feels firmer | N/A | 70.96% | 75.81% | 83.88% |
| 39. | My skin's elasticity feels | N/A | 69.35% | 75.80% | 80.64% |
| | improved | | | | |
| 40. | My skin feels nourished | N/A | 88.71% | 91.94% | 91.94% |
| 41. | My skin feels supple | N/A | 80.64% | 83.87% | 88.71% |
| 42. | My skin feels more resilient | N/A | 77.42% | 77.42% | 80.65% |

Although the foregoing refers to particularly preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

1. A cosmetic composition comprising: at least one superabsorbent polymer, at least one water soluble emollient, and at least one thickener, wherein the composition is an oil-in-water emulsion.

2. A cosmetic composition comprising: at least one superabsorbent polymer, at least one water soluble emollient, and at least two polymer thickeners.

3. The cosmetic composition of claim 1, wherein the at least one thickener comprises at least two polymer thickeners.

4. The cosmetic composition of claim 1, comprising at least about 60 mass % of water.

5. The cosmetic composition of claim 4, comprising from about 65 mass % to about 75 mass % of water.

6. The cosmetic composition of claim 1, wherein the at least one superabsorbent polymer comprises at least one of cross-linked sodium acrylates, acrylate grafted starches and mixtures thereof.

7. The cosmetic composition of claim 6, wherein the at least one superabsorbent polymer comprises sodium acrylates crosspolymer-2.

8. The cosmetic composition of claim 7, wherein the composition comprises from about 0.1 mass % to about 1.5 mass % of sodium acrylates crosspolymer-2.

9. (canceled)

10. The cosmetic composition of claim 1, wherein at least one water soluble emollient comprises at least two water soluble emollients.

11. The cosmetic composition of claim 1, wherein the at least one water soluble emollient comprises a water soluble microfluidized emollient composition.

12. The cosmetic composition of claim 1, wherein the at least one water soluble emollient comprises microfluidized squalane.

13. The cosmetic composition of claim 12, wherein the water soluble emollient composition comprises glycerin, water, squalane, and hydrogenated lecithin.

14. The cosmetic composition of claim 13 comprising from about 1 mass % to about 5 mass % of the water soluble emollient composition.

15. (canceled)

16. The cosmetic composition of claim 1, wherein the at least one water soluble emollient comprises an hydrophilic emollient containing at least one butter.

17. The cosmetic composition of claim 16, wherein the hydrophilic emollient comprises squalene, a mango seed butter and Orbignya oleifera seed oil.

18. The cosmetic composition of claim 17 comprising from about 0.5 mass % to about 1.5 mass % of the hydrophilic emollient.

19. The cosmetic composition of claim 1 comprising (a) from about 0.5 mass % to about 1.5 mass % of a hydrophilic emollient comprising squalene, a mango seed butter and Orbignya oleifera seed oil and (b) from about 2.5 mass % to about 3.5 mass % of a water soluble emollient composition comprising glycerin, water, squalane, and hydrogenated lecithin.

20. The cosmetic composition of claim 1 comprising at least two taurate based polymer thickeners.

21. The cosmetic composition of claim 20, wherein the at least two taurate based polymer thickeners comprise Ammonium Acryloyldimethyltaurate/VP Copolymer and Hydroxyethyl Acrylate/Sodium Acryloyldimethyl Taurate Copolymer.

22. The cosmetic composition of claim 20 comprising from 1.0 mass % to 2.2 mass % of the at least two taurate based polymer thickeners.

23. The cosmetic composition of claim 21, comprising from 0.4 mass % to 0.8 mass % of Ammonium Acryloyldimethyltaurate/VP Copolymer and from 0.8 mass % to 1.2 mass % of Hydroxyethyl Acrylate/Sodium Acryloyldimethyl Taurate Copolymer.

24. The cosmetic composition of claim 1, further comprising at least one hydrophobic emollient.

25. The cosmetic composition of claim 24, comprising from about 5 mass % to about 15 mass % of the at least one hydrophobic emollient.

26. The cosmetic composition of claim 1, further comprising at least one film former.

27. The cosmetic composition of claim 26, wherein the at least one film former comprises a film former comprising water, polyurethane, methylpropanediol, caprylil glycol and phenylpropanol.

28. The cosmetic composition of claim 26 comprising from 3 mass % to 5 mass % of the least one film former.

29. The cosmetic composition of claim 1 comprising (a) from about 0.3 mass % to about 0.7 mass % of sodium acrylates crosspolymer-2; (b) from about 0.5 mass % to about 1.5 mass % of a hydrophilic emollient comprising squalene, a mango seed butter and Orbignya oleifera seed oil; (c) from about 2.5 mass % to about 3.5 mass % of a water soluble emollient composition comprising glycerin, water, squalane, and hydrogenated lecithin; (d) from 0.4 mass % to 0.8 mass % of Ammonium Acryloyldimethyltaurate/VP Copolymer; and (e) from 0.8 mass % to 1.2 mass % of Hydroxyethyl Acrylate/Sodium Acryloyldimethyl Taurate Copolymer.

30. The cosmetic composition of claim 1, which is a silicone-free composition.

31. The cosmetic composition of claim 1, which is a PEG-free composition.

32. The cosmetic composition of claim 1, which is an alcohol-free composition.

33. A cosmetic method comprising applying the cosmetic composition of claim 1 to a keratinous surface.
